(19) United States
(12) Patent Application Publication
(10) Pub. No.: US 2010/0297587 A1
(43) Pub. Date: Nov. 25, 2010

(54) DENTAL CROWNS

(75) Inventor: Uri L. ZILBERMAN, Nes Ziona (IL)

Correspondence Address: THE NATH LAW GROUP, 112 South West Street, Alexandria, VA, 22314, US

(73) Assignee: URI-DENT LTD., Ashkelon (IL)

(21) Appl. No.: 12/793,426

(22) Filed: Jun. 3, 2010

Related U.S. Application Data
(63) Continuation-in-part of application No. 10/685,803, filed on Oct. 16, 2003.
Application No. 10/685,803 is a continuation-in-part of application No. PCT/IL2002/000310, filed on Apr. 16, 2002.
Application No. PCT/IL2002/000310 is a continuation of application No. 09/903,096, filed on Jul. 11, 2001, now Pat. No. 6,592,373.

(30) Foreign Application Priority Data
Apr. 17, 2001 (IL) 142657

(51) Int. Cl. A61C 5/10

(52) U.S. Cl. 433/223

(57) ABSTRACT

The present invention provides a dental crown formed of an elastically thermoplastic polymer material, said crown comprising: a tooth shaped top surface; and flexible side surfaces, at least one of which includes inwardly directed bottom portion.

This application is a Continuation-In-Part Application of U.S. patent application Ser. No. 10/685,803, filed on Oct. 16, 2003, which is a Continuation-In-Part Application of PCT International Application No. PCT/IL02/00310, filed on Apr. 16, 2002, which is a Continuation Application of U.S. patent application Ser. No. 09/903,096, filed on Jul. 11, 2001, now U.S. Pat. No. 6,592,373, which claims priority to Israeli Patent Application No. 142657, filed on Apr. 17, 2001, the content of each of which is hereby incorporated by reference in its entirety.

### FIELD OF THE INVENTION

The present invention is in the field of tooth prostheses and relates to permanent crowns.

### BACKGROUND OF THE INVENTION

The installation of permanent crowns to reconstruct a tooth, particularly human teeth, is a commonplace procedure generally accomplished in a sequence of steps over a prolonged period of time. Regarding the tooth shape replacement, it involves the preparation of a temporary crown and a permanent dental crown. The temporary crown is fitted onto a damaged tooth only for a limited period of time, typically from a few days to weeks. The permanent dental crowns then replace the temporary crown and serve as definitive tooth replacements and often remain in the patient's mouth for years or decades. For the dental crowns, it is also particularly important, in addition to the bite function, to ensure an attractive appearance of the tooth and of its surface. Ceramic has been used mainly as a suitable material for crowns intended as permanent crowns. It has good properties in respect of both functions. However, ceramic is an expensive material to machine.

Initially, impressions of the tooth to be reconstructed may be made to establish relationships with adjacent teeth. Then the tooth is ground to remove damaged portions and to provide a shape or “stump” which is adapted to receive the crown. Impressions or a mold is made from the shaped stump for transmittal to a dental laboratory where the permanent crown is molded. Prior to permanent fixation of the crown, a number of fittings are typically required.

In the meantime, while the crown is being prepared, it is necessary to keep the shaped tooth or stump protected from shock, further damage and exposure which could ultimately result in loss of the tooth. To accomplish this, a temporary crown or “crown form” is used to be installed on the shaped tooth immediately. Desirably, the crown form is quickly installed, well fitting, durable, easily removed and replaced and completely protective of the shaped tooth. Temporary crown forms or strip crowns are thin shells, commonly made of polycarbonate, typically used for holding and shaping a body of self curing plastic material. The thin shells become part of the temporary crown when filled with self-curing polymers. The gingival part of the shells is straight, rigid, and parallel and fits the gingival margins of the stump.

For restoration of anterior destroyed or broken tooth, instead of using a permanent crown, very thin, transparent and flexible shells exist that reproduce the final tooth size and shape. The shells are filled with semisolid composite material that flows during the insertion of the shell on the tooth and restore the broken part of the tooth. After placement the composite material is hardenned by aplying a special blue light to the material. Thus, the necessity that the shell be transparent and very thin so it will not affect the penetration of the blue light to the composite material, and will not affect the final size of the restoration after its removal. The crown form is removed from the restoration after curing of the composite material intended to remain on the tooth and restore it. The tooth to be restored using the composite material goes through clinical processes well known to general dentists: first, the outer enamel surface have to be prepared using a strong acid (a procedure known by the name of etching); the strong acid dissolves the enamel and creates micro-retentions on the surface; into those micro-retention grooves the composite enters and remain attached to the tooth surface after curing.

Conventional crown forms come in several types. One type is a shell of thin metal such as aluminum or copper. The other type is a synthetic polymeric resin (polycarbonate) form. These forms are durable, but large inventories of shapes and sizes must be maintained since they are difficult to adapt to any given shape or size. Further, the commercially available polycarbonate crown forms generally fail to make mesial-distal tooth contact properly because they are not adjustable in this direction without extensive modification.

As for artificial teeth and crowns, according to the conventional approach they are made from metal, porcelain, combinations of metal and porcelain. Crowns made of precious or semi-precious metal are expensive and the luster inherent in the metal does not match well with the existing teeth and body tissue and is therefore not desirable. Porcelain crowns have been utilized and are typically produced from casts from the actual tooth location. However, the firing of porcelain causes dimensional changes therein, and hence, the finished tooth may not properly fit the patient. Because this type of artificial tooth requires high technology of porcelain building and firing and must have high dimensional accuracy, the production cost thereof is very high. Combinations of metal and porcelain in which the porcelain is built and baked on the crown surface to shade the luster of the metal crown have been found to be complicated and also expensive to produce. All of the hereinabove described artificial teeth and crowns require a laboratory necessary for the fabrication of the tooth and crown. As a result, the procedure becomes expensive due to repeated visits to the dental office for refitting and additional procedural steps for the dentist.

In connection therewith, and also as an independent source of artificial crowns and teeth, is the technology built around newly-developed resins and epoxies or the like which are quick-setting. In this procedure, the resin is disposed in a mold and inserted in a tooth. After a period of time, the mold with partially set resin is removed from the patient's mouth and shaped by the dentist. Subsequent to continued hardening of the resin, it is cemented in place in the patient's mouth by a composite restorative material, and thereafter finished by polishing. When the mold is cut away it leaves the composite restorative material to serve as a crown. Unfortunately, the form, when removed, leaves the cast tooth undersized. The following U.S. patents and publications present some examples of the current state of the art: U.S. Pat. Nos. 4,129,946; 5,487,663; 5,624,261; 5,709,548; 6,106,295.

### GENERAL DESCRIPTION

The present invention seeks to provide a mass-produced, permanent pre-fabricated crown, which is made of a material enabling it to be tooth colored. The crown is particularly useful in pediatric dentistry for treatment of primary teeth and permanent molars having extensive carious lesions. The crown of the present invention is configured such that it can be directly applied to a tooth in a single stage procedure just by gluing the crown to a respective tooth, without a need for any adhesive resin and any restorative material.

When used herein, the term “permanent” means long lasting, as opposed to temporary dental restorations. Typically, temporary restorations are only designed to last six months or less, whereas the dental structure of the invention is a crown and is thus designed to last, at least theoretically, for the life of the patient, although these “permanent” restorations can be removed by a dentist if necessary. The term “crown” should be interpreted in its common meaning, namely referring to the whole tooth surface rather than selected portion(s) thereof.

The permanent crown of the present invention is prefabricated/preformed, namely is ready for directly mounting it onto a specific tooth. To this end, the crown is configured as a self-adjustable structure for wrapping/fitting the specific tooth, and eliminates a need for any additional filling materials except for a glue layer directly attaching the crown to the tooth.

There is thus provided by the present invention a dental crown, said crown being a prefabricated single-layer continuous structure having a predetermined shape corresponding to a shape of a tooth and being composed of a thermoplastic polymer material of a predetermined resilience, said crown comprising a top surface and side surfaces extending from said top surface towards a bottom of the structure, the side surfaces being flexible and at least one of the side surfaces including a curved bottom portion forming an undercut inwardly directing said bottom portion of the side surface, the predetermined resilience and shape of the prefabricated crown with the undercut being selected to provide elastic deformation of the crown such as to enable expansion, thereby enabling the crown to be used as a permanent crown. It should be noted that all the side surfaces may be formed with a curved undercut.

The crown of the present invention is elastically deformable, i.e., the crown resumes its initial shape after being fitted onto a patient's dentition. The curved undercut in the crown of the present invention is aimed at providing mechanical retention of the crown on teeth using buccal and lingual bulges that exist normally on primary and some permanent teeth. The undercut in the bottom portion of the inner surface of the flexible side surface(s) is aimed at enabling adjustment of the bottom portion of a crown to the specifically prepared tooth shape and preventing falling of the crown from the tooth.

The crown is made of elastically/flexible materials and therefore no changes of the original contour are observed after placement of the crown. The crown configuration eliminates a need to adjust its margins to the tooth margins, with no possibility to change occlusal morphology using hand instruments.

It should be understood that typically, for the treatment of permanent teeth, a crown of the tooth is reduced significantly on all its surfaces. The final configuration resembles a cylinder that all four walls converge slightly from the gingival aspect to the occlusal surface (the masticatory surface). In order to prepare the final crown an impression of the prepared tooth is taken and sent to a dental laboratory. The crown for the permanent tooth is prepared individually for each tooth. The final crown is prepared so that its internal surface is very close in its geometry (dimensions and shape) to the outer configuration of the prepared tooth. The retention of the crown to the tooth is based on vacuum formation between two almost identical cylinders. The preparation of the primary (deciduous) molar for crowns differs significantly from that of a permanent tooth, and accordingly the crown design is different. Accordingly, mainly reduced are the height of the tooth crown, the mesial and distal surfaces (surfaces close to the adjacent teeth), while the bulges on the facial and lingual surfaces are kept intact. The preformed crowns have the external surface similar to the primary molars, the crown is thin and prepared from a flexible material. Before the final adaptation of the crown, the crown is filled with cement and is placed over the tooth using a “snap-fit” procedure. The cement is used as the material that causes the internal surface of the crown to become cylindrical and to create the vacuum needed for the retention of the crown on permanent teeth.

When treating primary teeth, the relatively long time needed for the tooth preparation and the short time that a small child can seat in the dental chair should be taken into consideration. The idea of using the prefabricated/preformed crowns is aimed at minimally preparing the primary molars, in order to shorten the time, and to use other retentive methods for keeping the crown on the tooth. The preparation of the tooth is derived from the mechanical properties of retention of the crown. Primary molars have large bulges on the facial and lingual surfaces close to the gingiva. These bulges can be used as retentive mechanisms for the crown. The marginal aspect of the crown have to be elastic/flexible in order to jump over these bulges and have to be directed inwardly so that after the jump the margins will fit closely the crown surfaces. The margins of the crown have to be able to jump over the bulges, to expand during the snap-fit and to return to the original dimension in order to fit the surfaces of the tooth bellow the bulges. Therefore the surfaces of the crown have to be flexible and to bend inwardly at the cervical margins of the crown.

It should be understood that, typically, a cement layer between the crown and the tooth used for gluing the crown to the tooth in a snap-fit procedure mainly fills the minimal gap between the crown and the tooth and prevents saliva and food particles to enter between the crown and the tooth. It is not part of the crown and it should be kept to minimum. In the present invention, the cement has no retentive purposes, and it is not intended to become part of the crown in order to help in the retention of the crown. According to the conventional procedure of mounting a crown on a permanent tooth, a cement layer has a thickness not exceeding 0.3 mm, while the minimal required thickness of the crown is of the order of two millimeters or so. Generally, in the conventional procedure for permanent teeth, the cement layer is always much thinner than the crown thickness.

According to a preferred embodiment of the invention, the thermoplastic polymer material comprises a polymer selected from the following polymers: polyacetal, polyacrylate, polymethylmethacrylate (PMMA), polyamide, polyaryletherketone (PAEK), polyetherketone (PEK), polyetheretherketone (PEEK), polyetherimide (PEI), polyethersulfone (PES), polysulfone (PSU), and mixtures thereof. More preferably, the thermoplastic polymer is a homo- or co-polymer of acetal resin, polyetheretherketone (PEEK) or polymethylmethacrylate (PMMA).

According to another preferred embodiment of the invention, the thermoplastic polymer material further comprises at least one of the following: fibers, fillers, pigments and reinforcements. The fibers and fillers may be in their conventional or nano size.

A dental crown according to the present invention may be formed by several methods as one of ordinary skill in the art would be able to mass produce. Non-limiting examples of such methods includes injection molding, compression molding, stamp forming, vacuum forming, thermoforming, transfer molding composite flow molding, machining or the like.

According to a preferred embodiment of the invention, the dental crown is produced by mass production injection molding which comprises:

providing a multi-element mold; and

employing the multi-element mold to injection mold a dental crown from a thermoplastic polymer material.

Preferably, the multi-element mold includes an ejector, which is being operated to eject the molded crown following opening the multi-element mold.

### BRIEF DESCRIPTION OF THE DRAWINGS

The present invention will be understood and appreciated more fully from the following detailed description, taken in conjunction with the drawings in which:

FIG. 1 is a simplified pictorial illustration of a dental crown formed of acetal homopolymer;

FIG. 2 is a sectional illustration of the dental crown of FIG. 1, taken along lines II-II in FIG. 1; and

FIGS. 3A, 3B and 3C illustrate the operation of an apparatus for manufacturing a dental crown from acetal homopolymer resin in accordance with a preferred embodiment of the present invention.

### DETAILED DESCRIPTION OF THE INVENTION

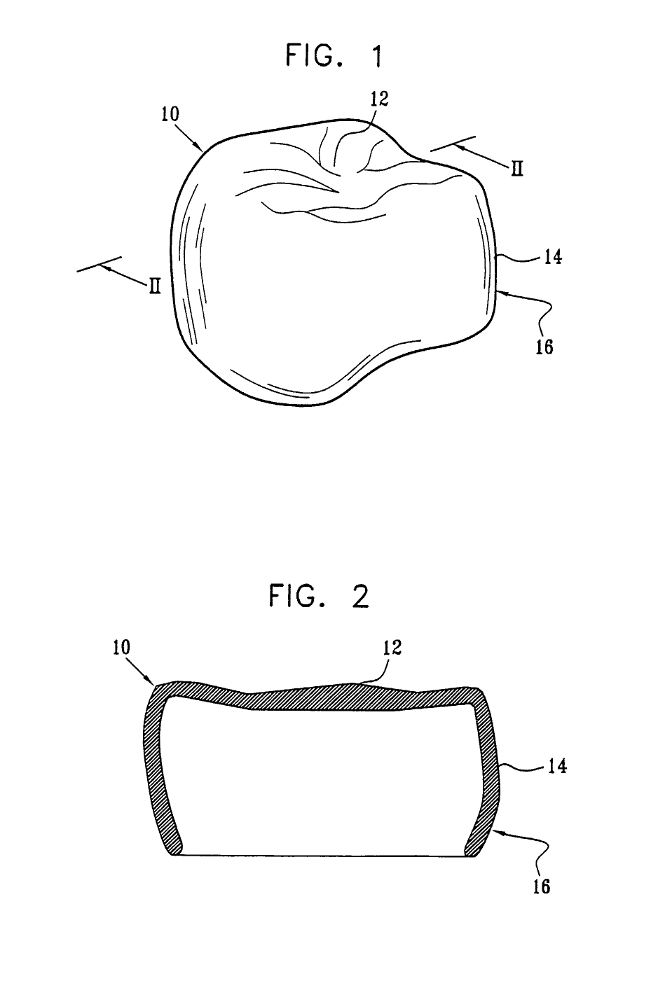

Reference is now made to FIG. 1, which is a simplified pictorial illustration of a dental crown formed of acetal homopolymer resin and to FIG. 2, which is a sectional illustration of the dental crown of FIG. 1, taken along lines II-II in FIG. 1.

As seen in FIGS. 1 and 2, there is provided in accordance with a preferred embodiment of the present invention prefabricated dental crown 10 comprising single-layer structure (i.e. one integral piece). The crown 10 is configured to have excellent dimensional stability and sufficient resilience (elasticity/flexibility and shape memory) and resistance to creep (force of mastication). Also, the crown's configuration is selected to provide the natural appearance of a vital tooth. Thus, the dimensions (thickness) and material(s) of the crown are selected to provide the desired resilience of the crown, namely ability to absorb energy when it is deformed elastically and then, upon unloading to have this energy recovered, or in other words, the maximum energy per unit volume that can be elastically stored (which is represented by the area under the curve in the elastic region in the Stress-Strain diagram of the specific material).

More specifically, it should be understood that the selection of the material and shape of the crown used in the present invention is not a trivial issue. On the one hand, the material to be used has to be elastic and resilient enough to provide to the crown the ability to resume its initial shape after being fitted onto a patient's dentition. On the other hand, the material and the configuration of the crown have to be selected to make the crown strong enough to meet the requirements of the permanent crowns, namely support masticatory forces up to about 500-700N. It would be understood by a person skilled in the art that in order to meet the requirement for a high mechanical strength (tensile strength), which is a standard requirement for a permanent crown, a thickness of the crown should be appropriately selected. The thickness need not and practically should not be the same along the crown, but rather is higher at the top surface (e.g. 1.2 mm), and thinner at the bottom portion of the side surfaces (e.g. 0.3 mm). Thus, for the purposes of the invention, the material for the crown and the undercut geometry (bending angle) are selected together with the thickness conditions.

The requirement for desired resilience can thus be reached by selecting the material as defined by a stress-strain curve of the material indicating that the yield point occurs when the material reaches a strain of almost 15%. Generally, the stress-strain curve is a graphical representation of the relationship between stress, derived from measuring the load applied on the sample, and strain, derived from measuring the deformation of the sample, i.e. elongation, compression, or distortion. The resilience property is thus defined by the yield point of the material, which in turn is defined by the transition from elastic behavior (reversible deformation of a material) to plastic behavior of a material (deformation of a material undergoing non-reversible changes of shape in response to applied forces).

A preferred material for the crown is acetal homopolymer resin (DELRIN®) which is commercially available from DuPont.

As can be readily seen in FIGS. 1 and 2, the dental crown 10 is formed with a generally conventionally tooth shaped top surface 12 and depending side surfaces 14. The side surfaces are flexible. At least one of the side surfaces is configured to define an undercut 16 made as a gradually curved bottom portion of the side surface, which curve (bent portion) is designed according to the shape of a tooth-type for which the crown is pre-fabricated. The undercut has an inwardly arcuate and angled taper toward the gingival end.

The undercut-relief of the flexible side surface (at least one of the flexible side surfaces 14) and the resilience of the crown material allows the crown to be expandable for mounting on the tooth and to be then retracted to fit the tooth, and thus enables the dental crown 10 to be used for treatment of primary teeth and permanent molars. Prefabricated crown 10, i.e. readily mountable onto a damaged tooth, can then mounted by conventional methods, such as through the use of dental cement in the mouth of a patient, typically a child, as part of treatment of primary teeth and permanent molars having extensive carious lesions. More specifically, the inner surface of the crown is coated by glue, then the crown is brought to the tooth and its side surfaces are expanded to enable their mount onto the tooth, and then released to fit the tooth. It is a particular feature of the prefabricated crown 10 to be of a color which generally matches that of the patient's teeth.

The crown of the present invention is characterized by high tensile strength, high impact resistance and stiffness, excellent fatigue endurance and resistance to moisture, excellent dimensional stability and sufficient resilience and resistance to creep. It has the natural appearance of a vital tooth.

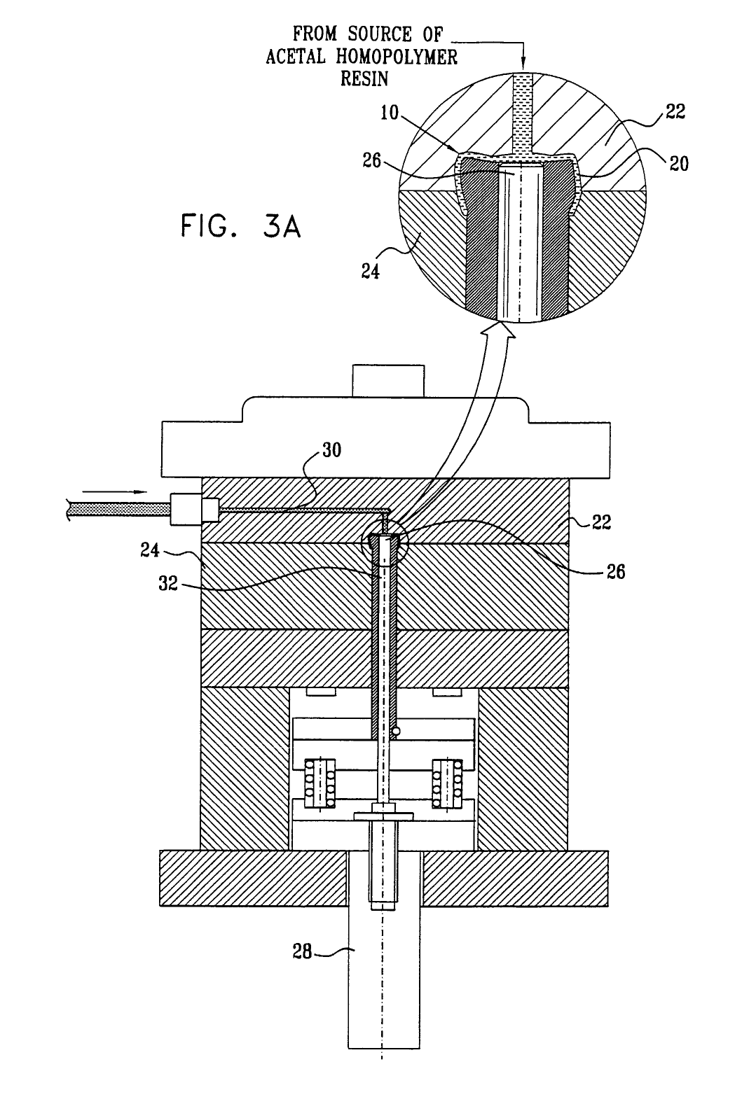

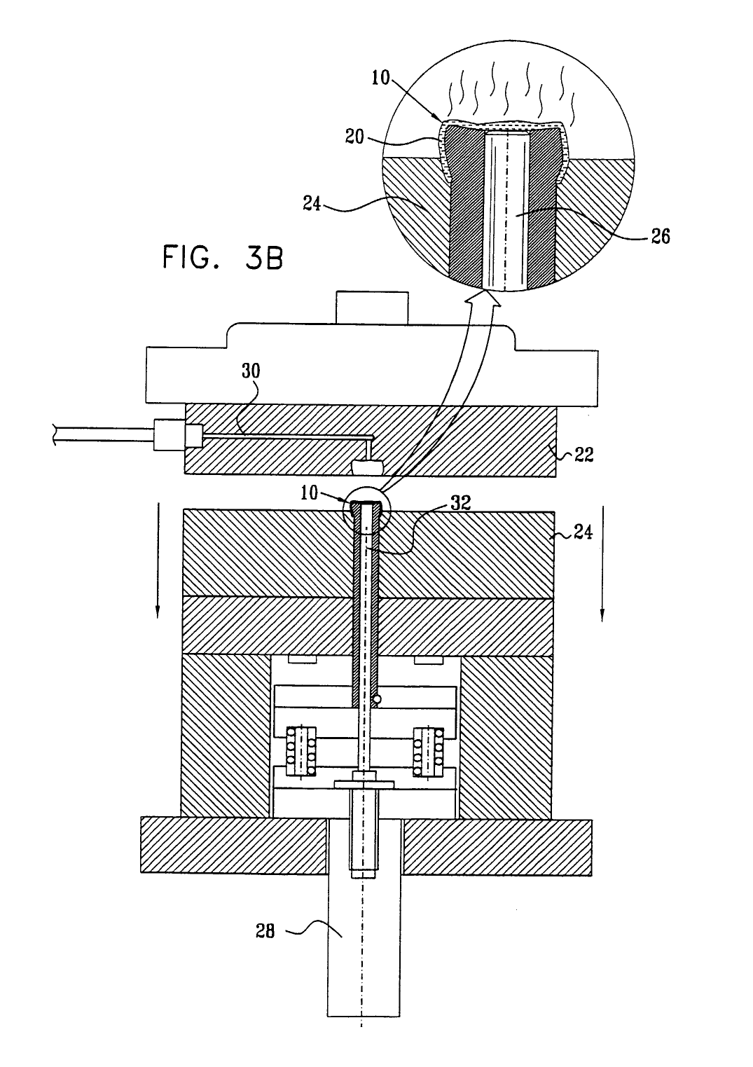

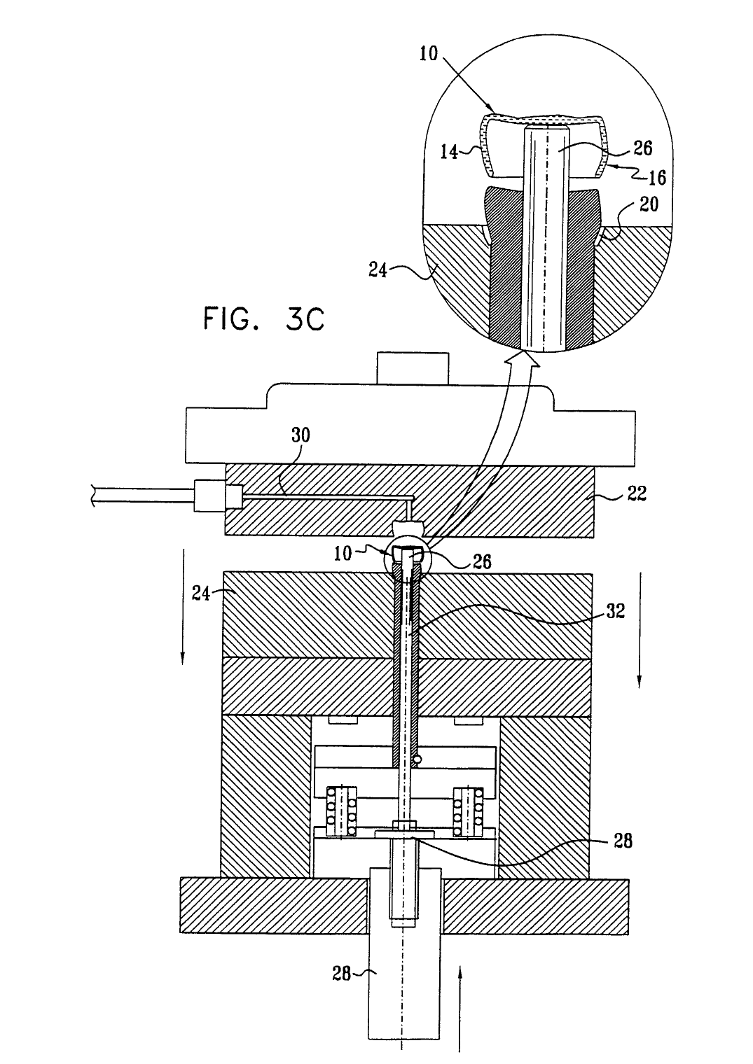

Reference is now made to FIGS. 3A, 3B and 3C, which illustrate the operation of an apparatus for manufacturing a dental crown from acetal homopolymer resin in accordance with a preferred embodiment of the present invention.

As seen in FIGS. 3A, 3B and 3C, the crown 10 is molded in a mold cavity 20 which is defined by a top mold element 22, a bottom mold element 24 and an ejector 26. The ejector 26 forms part of an internal mold element 32.

FIG. 3A shows the stage of molding when the top mold element 22 lies in tight engagement with the bottom mold element 24 and the ejector 26. The dental crown 10, which is fabricated on the ejector 26, is formed by the injection of acetal homopolymer resin material from a source of acetal homopolymer resin (not shown) into the mold cavity 20, via a channel 30 cut in the top mold element 22.

FIG. 3B shows an initial release stage wherein the bottom mold element 24 is separated from the top mold element 22, thus permitting removal of the molded crown 10 from cavity 20.

FIG. 3C shows an ejection stage wherein ejector 26, driven by a piston 28 moves upwardly relative to bottom mold element 24 and pushes crown 10 out of cavity 20. Due to the resilience of the depending side surfaces 14, the action of the ejector 26 is able to disengage the internal mold element 32 from the crown 10 notwithstanding the presence of undercut 16.

It will be appreciated by persons skilled in the art that the present invention is not limited by what has been particularly shown and described hereinabove. Rather the scope of the present invention includes both combinations and subcombinations of the various features described hereinabove as well as variations and modifications which would occur to persons skilled in the art upon reading the specification and which are not in the prior art.

1. A dental crown, said crown being a prefabricated single-layer continuous structure having a predetermined shape corresponding to a shape of a tooth and being composed of a thermoplastic polymer material of a predetermined resilience, said crown comprising a top surface and side surfaces extending from said top surface towards a bottom of the structure, the side surfaces being flexible and at least one of the side surfaces including a curved bottom portion forming an undercut inwardly directing said bottom portion of the side surface, the predetermined resilience and shape of the prefabricated crown with the undercut being selected to provide elastic deformation of the crown such as to enable expansion of the crown to mount it onto the tooth and retraction back to fit the shape of said tooth, thereby enabling the crown to be used as a permanent crown.

2. A dental crown according to claim 1, wherein said thermoplastic polymer material comprising a polymer selected from polyacetal, polyacrylate, polymethylmethacrylate (PMMA), polyamide, polyaryletherketone (PAEK), polyetherketone (PEK), polyetheretherketone (PEEK), polyetherimide (PEI), polyethersulfone (PES), polysulfone (PSU), and mixtures thereof.

3. A dental crown according to claim 2, wherein said thermoplastic polymer is a homo- or co-polymer of acetal resin, polyetheretherketone (PEEK) or polymethylmethacrylate (PMMA).

4. A dental crown according to claim 1, wherein said thermoplastic polymer material further comprising at least one of the following: fibers, fillers, pigments and reinforcements.

5. A dental crown, said crown being configured as a prefabricated single-layer continuous structure having a predetermined shape corresponding to a shape of a tooth and being composed of acetal resin of a predetermined resilience, said crown comprising a top surface and side surfaces extending from said top surface towards a bottom of the structure, the side surfaces being flexible and at least one of the side surfaces including a curved bottom portion forming an undercut inwardly directing said bottom portion of the side surface, the predetermined resilience and shape of the prefabricated acetal resin crown with undercut providing elastic deformation of the crown such as to enable expansion of the crown to mount it onto the tooth and retraction back to fit the shape of said tooth thereby enabling the crown to be used as a permanent crown.

6. A dental crown, said crown being configured as a prefabricated single-layer continuous structure having a predetermined shape corresponding to a shape of a tooth and being composed of a predetermined material having a yield point corresponding to a strain of at least 15%, thereby providing a predetermined resilience of the crown, said crown comprising a top surface and side surfaces extending from said top surface towards a bottom of the structure, the side surfaces being flexible and at least one of the side surfaces including a curved bottom portion forming an undercut inwardly directing said bottom portion of the side surface, the predetermined resilience and shape of the prefabricated crown with undercut providing elastic deformation of the crown such as to enable expansion of the crown to mount it onto the tooth and retraction back to fit the shape of said tooth thereby enabling the crown to be used as a permanent crown.

7. A dental crown according to claim 1, formed by injection molding.

8. A dental crown according to claim 7, produced by mass production injection molding comprising:
providing a multi-element mold; and
employing the multi-element mold to injection mold a dental crown from a thermoplastic polymer material.

9. A dental crown according to claim 8, wherein said multi-element mold includes an ejector, which is being operated to eject the molded crown following opening the multi-element mold.

10. A dental crown according to claim 1, formed by compression molding.

11. A dental crown according to claim 1, formed by machining.
